(19) United States
(12) Patent Application Publication
(10) Pub. No.: US 2025/0375246 A1
(43) Pub. Date: Dec. 11, 2025

(54) AUTOMATED ARTHOPLASTY PLANNING WITH MACHING LEARNING

(71) Applicant: Think Surgical, Inc., Fremont, CA (US)

(72) Inventor: Joel Zuhars, Fremont, CA (US)

(73) Assignee: Think Surgical, Inc., Fremont, CA (US)

(21) Appl. No.: 18/876,819

(22) PCT Filed: Jun. 30, 2023
(86) PCT No.: PCT/US2023/026661

§ 371 Date: Dec. 19, 2024

Related U.S. Application Data
(60) Provisional application No. 63/357,096, filed on Jun. 30, 2022.

(51) Int. Cl. A61B 34/10; A61B 34/00; A61F 2/46

(52) U.S. Cl. CPC A61B 34/10; A61B 34/25; A61F 2/461; A61F 2002/4633

(57) ABSTRACT

A surgical planning system for planning a surgery based on experience of a historical user is provided that includes a computer operatively coupled to a display for displaying a graphical user interface (GUI) and a processor configured to execute the planning software. The computer or the software generate surgical plans based on the set of characteristics of the bone image and the recognized patterns of the planning model corresponding to experience of the first historical user. A computerized method for defining implant positioning data relative to a bone image or evaluating the surgical plans are also provided. A method of performing surgery on a subject is also provided according to a so developed surgical plan.

### RELATED APPLICATIONS

This application claims priority benefit of U.S. Provisional Application Ser. No. 63/357,096 filed 30 Jun. 2023; the contents of which are hereby incorporated by reference.

### TECHNICAL FIELD

The present invention generally relates to the field of computer-aided surgical planning, and more specifically to a computerized method to plan an arthroplasty procedure using machine learning.

### BACKGROUND

Joint arthroplasty is a surgical procedure in which the articulating surfaces of bones are replaced with prosthetic components, or implants. For example, in total knee arthroplasty (TKA) the worn or damaged cartilage and bone on the distal femur and proximal tibia are removed and replaced with synthetic implants, typically formed of metal or plastic, to create new joint surfaces.

Computer-assisted surgical devices are popular tools to plan and precisely execute a joint arthroplasty procedure to improve long term clinical outcomes and increase the survival rate of the implants. A computer-assisted surgical system generally includes two components: i) planning software for positioning and orientating (posing) an implant image with respect to a bone image to designate a position and orientation (“POSE”—“POSE” may also refer to “position and orient” where applicable) for the implant when mounted on the bone; and ii) a computer-assisted surgical device for removing bone to form surfaces (“cut surfaces”) on the remaining bone at locations where the implant when mounted on the cut surfaces is in the designated POSE. The implant may include one or more contact surfaces that contact the cut surfaces to mount the implant on the remaining bone, either directly or indirectly (e.g., via a cement interface).

With reference to FIG. 1, conventional planning software may provide a bone image “BI” in the form of a three-dimensional (3-D) model of the patient's bone (i.e., a bone model) generated from an image data set (e.g., computed tomography (CT) or magnetic resonance imaging (MRI)) of the patient. The implant image 12 may be provided in the form of a 3-D computer-aided-design (CAD) model of the implant (i.e., an implant model), where a plurality of manufacturers' implant models may be pre-loaded in the planning software. FIG. 1 depicts a femoral implant model POSED with respect to a femoral bone model for a TKA procedure. During planning, an implant image 12 is positioned and oriented (POSED) with respect to the patient's bone image “BI” to designate a POSE for the implant when mounted on the bone. The process of “generating a surgical plan” refers to the process of determining a POSE for an implant with respect to a bone, which may be determined by posing an implant image 12 with respect to a bone image “BI”. A “surgical plan” 10 refers to a planned POSE for an implant with respect to a bone, which may be represented or provided as an implant image 12 POSED with respect to a bone image “BI” as shown in FIG. 1. It should be appreciated, that the process of “generating a surgical plan” may also refer to the process of determining a POSE for an implant when mounted on the bone, which may or may not require the use of images. The surgical plan 10 may be saved and transferred to a computer-assisted surgical device for removing bone to form cut surfaces on the remaining bone such that the implant when mounted on the cut surfaces is in the designated POSE.

One particular problem with conventional planning software is the need to perform a number of planning steps manually. For instance, a user (e.g., a surgeon, a case planner) may have to manually identify a plurality of anatomical landmarks on the bone, where those anatomical landmarks are important for determining anatomical references (e.g., a bone's mechanical or longitudinal axis, angles of anatomical structures with respect to one another) required to plan a POSE for an implant model relative to a bone model according to clinically acceptable standards (e.g., neutral alignment to the mechanical axis). The planning software may also require the user to manually move the implant model with respect to the bone model and the anatomical references until a final POSE for the implant model with respect to the bone model is obtained. These manual steps are both time-consuming and prone to error.

Planning software has been developed to reduce the number of these manual planning steps, such as the planning software described in U.S. patent application Ser. No. 16/080,735, filed Aug. 29, 2018, assigned to the assignee of the present application, and incorporated by reference herein in its entirety. In certain cases, a secondary user (e.g., a case planner) may generate surgical plans for primary users (e.g., surgeons). Each primary user may have a specific strategy for posing an implant image with respect to a bone image. Such strategies may be based on years of learned experience. For example, FIG. 2 depicts a table of planning preferences for a TKA femoral component from two different surgeons, which correspond to each surgeon's planning strategy (which may also be considered the surgeon's planning philosophy). Surgeon 1 may prefer to use implant 1 (e.g., manufacturer A's implant), a posterior stabilized (PS) technique, a neutral mechanical axis alignment for coronal alignment, an internal-external rotational alignment of 3° from the posterior condylar axis (PCA), 8-9 millimeters (mm) of distal resection, and 8-9 mm of posterior resection. Surgeon 2 may prefer to use implant 2 or implant 3 (e.g., manufacturer B's implant or manufacturer C's implant), a PS or cruciate retaining (CR) technique, a neutral mechanical axis alignment or native kinematic alignment for coronal alignment, an internal-external rotational alignment of parallel to the transepicondylar (TEA) axis or native from the PCA, 7.5-8.5 mm in distal resection, and 7.5-8.5 mm in posterior resection.

When a case planner is assigned a patient case from a particular surgeon, the case planner references the table in FIG. 2 to POSE an implant image with respect to the patient's bone image according to that surgeon's preferences. However, a case planner is only able to consider a limited number of factors that contribute to that surgeon's preferences, and is not able to fully consider all of relevant factors that may be considered by the actual surgeon based on that surgeon's experience with hundreds, if not thousands, of previously planned and executed cases. This surgical plan is provided to the surgeon, who may adjust the POSE of the implant image based on their experience, planning strategy, and patient specific factors. For instance, the surgeon may adjust the POSE of the implant image based on at least one of the following representative factors: (i) the patient's unique bone geometry; (ii) the bone geometry of adjacent bones; (iii) bone quality or density; (iv) the planned POSE for other implant components (e.g., the POSE of the tibial implant POSE on the tibia, implant liner sizes); (v) the surrounding anatomy (e.g., presence of osteophytes, location of tendons and ligaments); (vi) clinical outcomes of patient cases with similar bone geometry; (vii) patient's body mass index (BMI); (viii) ease of access to the surgical site (e.g., incision size); (ix) the bilateral anatomy and any previous surgeries (e.g., a TKA in the bilateral knee); (x) the surgeon's planning strategy; and (xi) any other patient specific factors or surgeon preferences. For example, a case planner may be assigned a patient case from surgeon 2 and generate a surgical plan according to the following preferences: implant 2, PS technique, neutral coronal alignment, parallel to the TEA in rotational alignment, 8 mm of distal resection and 8 mm in posterior resection. Note, the case planner may have used an educated guess for the amount of distal resection and posterior resection since surgeon 2 provides the case planner with a range of values in Table 1 of FIG. 2. Surgeon 2 may receive and review this surgical plan and make the following adjustments to the surgical plan: implant 3, a CR technique, neutral coronal alignment, parallel to the TEA in rotational alignment, 7.6 mm distal resection, and 8.2 posterior resection. Surgeon 2 may have noticed this patient has a very particular bone geometry or bone quality that warranted these changes. It is possible that a surgeon may completely deviate from their traditional planning strategy for a particular patient based on the factors identified above. As such, it becomes apparent that the case planner (i.e., the secondary user) simply lacks the experience and knowledge of a surgeon (i.e., the primary user) to generate a surgical plan like the surgeon.

Furthermore, a less experienced surgeon may not have adopted their own planning strategy or are still training among other surgeons to fine-tune their planning/surgical skills. This surgeon simply lacks the experience to consider all of the above factors to generate an optimal surgical plan, where it may take a considerable amount of time and effort to obtain the experience of a seasoned surgeon. There may also be a situation where an experienced surgeon decides to change their planning strategy to align with another surgeon's planning strategy for a given procedure. This can likewise take a considerable amount of time and effort for that surgeon to master the other surgeon's planning strategy.

Thus, there is a need for a system and method for automatically generating a surgical plan for a first user as if the surgical plan was generated and finalized by a second user using the second user's planning strategy and based on the second user's experience.

### SUMMARY OF THE INVENTION

A surgical planning system for planning a surgery based on experience of a historical user is provided that includes a computer operatively coupled to a display for displaying a graphical user interface (GUI) and a processor configured to execute the planning software. The computer is operative to:
receive a bone image and display the bone image on the GUI;
receive, via the GUI, a selection of a planning model corresponding to experience of a first historical user, the planning model based on recognized patterns in a collection of planned cases of the first historical user;
analyze the bone image of the patient to determine a set of characteristics of the bone image;
compare the set of characteristics of the bone image to bone characteristics of the collection of planned cases; and
generate a surgical plan comprising implant positioning data defined with respect to the bone image based on the set of characteristics of the bone image and the recognized patterns of the planning model corresponding to experience of the first historical user.

A surgical planning system for planning a surgery based on experience of a historical user, the system is also provided that includes a computer having a processor configured to execute planning software. The planning software operative to:
receive a bone image;
receive a selection of a planning model corresponding to experience of a first historical user, the planning model based on recognized patterns in a collection of planned cases of the first historical user;
analyze the bone image to determine a set of characteristics of the bone image;
compare the set of characteristics of the bone image to bone characteristics of the collection of planned cases; and
generate a surgical plan comprising implant positioning data defined with respect to a bone based on the set of characteristics of the bone image and the recognized patterns in the planning model.

A computerized method for positioning an implant image relative to a bone image, is also provided in which a first planning model and a second planning model are provided where the first planning model is generated with machine learning using first historical planning data from a first historical user and the second planning model is generated with machine learning using second historical planning data from a second historical user. A selection of the first planning model or the second planning model is then provided. The selected first planning model or second planning model is then executed with an input including a bone image to automatically define implant positioning data with respect to the bone image.

A computerized method for evaluating surgical plans as detailed above is provided that executes the selected first planning model with an input including a bone image to generate a first surgical plan including a first position for a first implant image relative to the bone image; and executes the selected second planning model with the input to generate a second surgical plan including second implant positioning data defined with respect to the bone image. The first surgical plan and the second surgical plan are then displayed for comparison.

A method of performing surgery on a subject is also provided that includes generating a surgical plan with the surgical planning system and performing the surgery on a subject according to the surgical plan.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 depicts a femoral implant model POSED with respect to femoral bone model for a TKA procedure;

FIG. 2 depicts a table of planning preferences for planning a POSE for a TKA femoral implant relative to a bone from two different surgeons, which correspond to each surgeon's planning strategy;

FIG. 3 illustrates a method for generating a trained planning model of a historical user and automatically generating a surgical plan according to a historical user's planning strategy according to embodiments of the present invention;

FIGS. 4A-4D show bone images annotated for training a machine learning model according to embodiments of the present invention.

FIG. 5 depicts an embodiment of a surgical planning workstation GUI according to embodiments of the present invention;

FIG. 6 shows an implant posed on a patient bone image according to embodiments of the present invention;

FIG. 7 shows a surgical plan comparison display on a GUI according to embodiments of the present invention;

FIG. 8 shows a 3-D bone model of a patient bone with an error map generated using a planning model according to embodiments of the present invention

FIG. 9 depicts a bone image of a patient that includes a metal artifact;

FIG. 10 shows a plurality of cut paths associated with an implant image according to embodiments of the present invention; and

FIG. 11 depicts a plurality of boundaries of a surgical plan generated using a planning model according to embodiments of the present invention.

### DESCRIPTION OF THE INVENTION

The present invention has utility as a system and method for automatically generating a surgical plan for a first user as if the surgical plan was generated and finalized by a second user using the second user's planning strategy and based on the second user's experience.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The degree of surgical outcome success is dependent on the surgeon's ability to devise and implement the best surgical plan for a particular patient's needs. However, this may not be achievable in view of inherent limitations in a surgeon's ability to adapt past practice experiences to a patient's particular bone geometries and corrective needs when selecting among a myriad of combinations of clinically acceptable standards and numerous available implant lines, implant types, and configurations. This suggests that there are problems that have not been addressed with previous medical procedures and related innovations.

According to embodiments, machine learning is used to simulate surgical planning like a particular surgeon based on a collection of that surgeon's planned cases. For example, using machine learning to simulate surgical planning like a particular surgeon based on a collection of that surgeon's planned cases and then having another surgeon, for example a less experienced surgeon, able to select from a library of experienced surgeons to have a surgical plan automatically generated as if the selected experienced surgeon had created the surgical plan. According to embodiments, a collection of a particular experienced surgeon's (i.e. a historical user's) planned cases are used as input into a computer and analyzed using machine learning to recognize patterns in the historical user's planned cases. For example, the machine learning may recognize how that particular historical user reacts when presented with a bone having a particular geometry, density, or quality and the decisions that user makes for positioning, and/or selecting, an implant. The machine learning is able to analyze an unlimited number of a historical user's planned cases, recognizing more and more patterns and understanding the user's decision making process and experience better and better with every additional case that is analyzed. As such, the machine learning is able to understand a historical user's planning strategy (or philosophy) far better than any existing system or third-party case planner referencing a preferences table, such as that shown in FIG. 2. In addition, the machine learning is able to identify and learn from the smallest deviations between one surgical plan from another, including the minutest differences in bone geometries and other characteristics which affect the resulting POSE of an implant image with respect to a bone image. These small deviations may be impossible for any other human, other than the experienced surgeon, to identify. The output of the machine learning's study of the historical user's planned cases is a planning model. According to embodiments, the resulting planning model is used in conjunction with a surgical planning software to plan a present surgery case based on the experience of the historical user upon which the planning model is based. The result being a surgical plan for a present case, planned the same way the historical user would plan the case. This is advantageous in that a single person can only plan so many surgeries given the time and detail required; however, the present invention allows a computer to automatically plan a surgery just like the historical user would. Accordingly, the system and method of the present invention is able to plan far more surgeries than a single person ever could. In addition, the present invention provides the ability to consistently generate a surgical plan like an experienced historical user, which results in improved, and more consistent, clinical outcomes. This improvement and consistency is enhanced when combined with the use of a computer-assisted surgical device capable of accurately executing the surgical plan on the patient.

According to embodiments, a less experienced surgeon, or any other user (e.g., a case planner), may use the inventive system and method to select a planning model of an experienced historical user (e.g., their favorite surgeon) to generate a surgical plan like that historical user, thereby leveraging the experience of the historical user. This less experienced surgeon is thereby able to perform a procedure on a patient with results that may mimic that of the experienced user. For example, the less experienced surgeon, with the assistance of a computer-assisted surgical device, may form cut surfaces on a patient's bone at locations where an implant when mounted on the cut surfaces is positioned according to how the experienced user would position and mount the implant on the cut surfaces of the bone. The less experienced surgeon also learns from the experience of the historical user, even if the less experienced surgeon has never met the experienced surgeon. According to embodiments, the less experienced surgeon is also able to generate a first surgical plan using a first planning model based on a first historical user and generate a second surgical plan using a second planning model based on a second historical user within minutes. The less experienced surgeon is then able to compare the generated surgical plans to compare the prosed techniques. Such a comparative tool has not been available previously. The present invention accordingly allows less experienced surgeons to be trained like an existing experienced surgeon.

According to certain inventive embodiments, the system and method includes automated planning software that is customized with a particular planning model, or a particular set of planning models, to plan present surgical cases as if a particular historical user was planning the surgery. Each planning model may be provided as a part of an inventive system or may be a plug-in component for an existing planning software to give an existing planning software an enhanced feature of planning a surgery like a historical user.

As used herein, the term “surgical plan” refers to a planned POSE for an implant with respect to a bone, and may be represented or provided as any one of the following, or a combination thereof: (a) the POSE for the cut surfaces to be formed on the remaining bone for mounting an implant thereon in a planned POSE; (b) raw POSE data for an implant with respect to the bone such as the femoral distal resection orientation and amount, the femoral posterior resection orientation and amount, the femoral anterior resection orientation and amount, the femoral chamfer resections orientation and amount, and the tibial proximal resection orientation and amount; (c) the planned clinical alignment data for the implant with respect to the bone such as the planned coronal alignment (e.g., neutral alignment), the planned rotational alignment (e.g., parallel to TEA), the amount of femoral distal resection, the amount of femoral posterior resection, the amount of anterior resection, the amount of tibial proximal resection; and/or (d) an implant image POSED with respect to a bone image. The surgical plan may further include additional data, such as the implant line of the implant image (e.g., the implant image corresponds to an implant manufactured by manufacturer A), an implant size associated with the implant image, patient identifier data, patient medical history, patient demographics, the POSE of other implants or implant components with respect to the bone image or with respect to a bone image of an adjacent bone. The surgical plan may further include software instructions (e.g., cut paths, end-effector orientations, end-effector feed-rates, virtual boundaries, virtual planes) for directing a computer-assisted surgical device to assist in the removal of bone to form one or more cut surfaces on the remaining bone. Examples of such computer-assisted surgical devices include tracked surgical instruments, robotic hand-held devices, serial-chain robots, bone mounted robots, parallel robots, or master-slave robots, as described in U.S. Pat. Nos. 5,086,401; 6,757,582; 7,206,626; 8,876,830; and 8,961,536; U.S. Patent Publication No. 2013/0060278; and PCT Patent Publication Nos. PCT/US2021/031703; and PCT/US2020/062686, all of which patents and patent applications are incorporated herein by reference. The surgical robot may be active (e.g., automatic/autonomous control), semi-active (e.g. a combination of automatic and manual control), haptic (e.g., tactile, force, and/or auditory feedback), and/or provide power control (e.g., turning a robot or a part thereof on and off).

An implant image may be a two-dimensional (2-D) or 3-D representation of an implant. For example, the implant image may be a 3-D CAD model of an implant, a laser scanned image of an implant, a point cloud of the outer surface of the implant, a planar 2-D image, or a set of planar 2-D images (e.g., a series of cross-sectional 2-D images, two or more orthogonal 2-D planar images). The bone image may be a 2-D or 3-D representation of the bone. By way of example, the bone image may be one or more of the following: an image data set of the bones (e.g., an image data set acquired via computed tomography (CT), magnetic resonance imaging (MRI), ultrasound, x-ray, laser scan, etc.); three-dimensional (3-D) bone models, which may include a virtual generic 3-D model of the bone, a physical 3-D model of the bone, a virtual patient-specific 3-D model of the bone generated from an image data set of the bone; and a set of data collected directly on the bone intra-operatively commonly used with imageless CAS devices (e.g., laser scanning the bone, digitizing the bone (e.g., “painting” the bone with a digitizer), generating a point cloud of the bone).

A model of machine learning operative herein is an artificial neural networks (ANN) that uses inputs and training sets of data to predict outcomes by identifying patterns within the training data. The application of ANN to medical imaging data is known. AS Lundervold et anon. Zeitschrift fur Medizinische Physik. 2018; 29:102-127. An ANN is well suited for use in the present invention as it allows for factors and the respective results to be entered into a computer for data analysis to determine which factors are necessary to predict certain outcomes. As a result, an inventive system develops a network of neurons without a human inputting a hypothesis and testing thereof. The patterns thus emerge naturally, and the output of the analysis can be used to predict future outcomes.

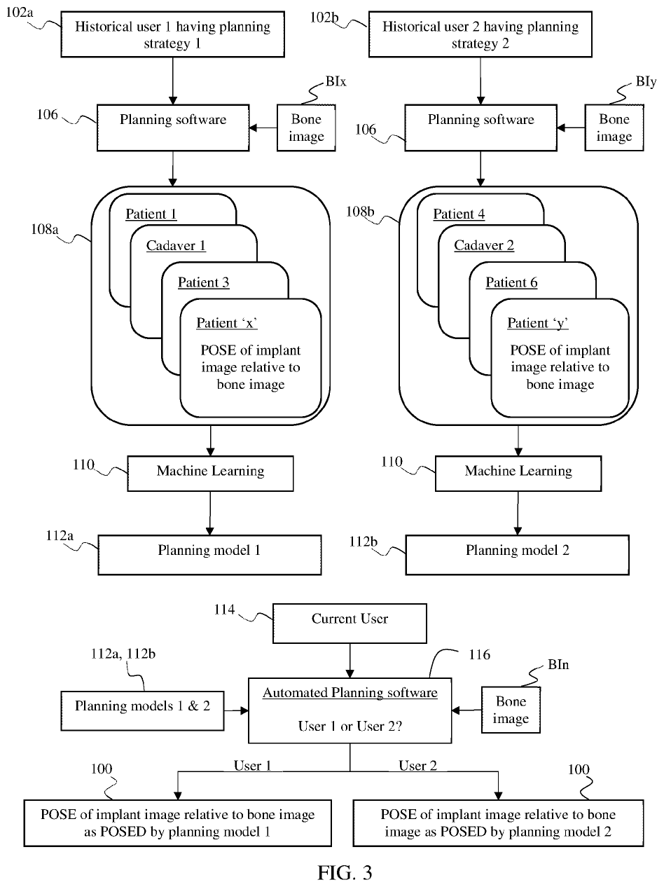

FIG. 3 illustrates a particular embodiment of a method for generating a trained planning model of a historical user and automatically generating a surgical plan according to a historical user's planning strategy. A first historical user 102a (e.g., a first surgeon, a first case planner, a first medical technician) may have a first planning strategy, such as the planning strategy outlined in row 2 of the table shown in FIG. 2. A second historical user 102b (e.g., a second surgeon, a second case planner, a second medical technician) may have a second planning strategy, such as the planning strategy outlined in row 3 of the table shown in FIG. 2. A collection of surgical plans (108a, 108b) from different cases (e.g., patient cases, cadaver cases) are generated, or have been generated, by each historical user (102a 102b) using planning software 106 and at least one bone image “BI” that was acquired for each case. Historical user 1 may have generated the collection of surgical plans 108a on a first set of bone images “BIx” (e.g., the collection of surgical plans include a first surgical plan generated with a bone image of patient 1's bone, a second surgical plan generated with a bone image of patient 2's bone, etc.), and historical user 2 may have generated a collection of surgical plans 108b on a second set of bone images “BIy”, where the first set of bone images “BIx” may by unique with respect to the second set of bone images “BIy”, the same set, or partially the same set. An example of planning software 106 for generating each surgical plan in the collection of surgical plans (108a, 108b) is described in U.S. patent application Ser. No. 16/080,735. Each collection of surgical plans (108a, 108b) is provided as an input into a machine learning program 110 to output a planning model (112a, 112b) for each historical user (102a, 102b). For example, the machine learning program outputs planning model 1, 112a, based on the collection of surgical plans 112a from historical user 1. Each planning model (112a or 112b) is therefore trained to generate a surgical plan according to each historical user's planning strategy, respectively. The planning models (112a, 112b) account for numerous factors a historical user may consider when generating a surgical plan, such as: (i) a patient's unique bone geometry; (ii) a bone geometry of adjacent bones; (iii) bone quality; (iv) a planned POSE for other implant components (e.g., the POSE of the tibial implant POSE on the tibia, implant liner sizes); (v) surrounding anatomy (e.g., presence of osteophytes, location of tendons and ligaments); (vi) clinical outcomes of patient cases with similar bone geometry; (vii) patient's body mass index (BMI); (viii) ease of access to the surgical site (e.g., incision size); (ix) the bilateral anatomy and any previous surgeries (e.g., a TKA in the bilateral knee); (x) the historical user's planning strategy; and (xi) any other patient specific factors or surgeon preferences. Each planning model (112a, 112b) may be incorporated into automated planning software 116 or provided as an optional plug-in to existing planning software 106. The planning models (112a, 112b), using a new bone image “BIn” as input, generates surgical plans the same way the historical user would generate the surgical plan.

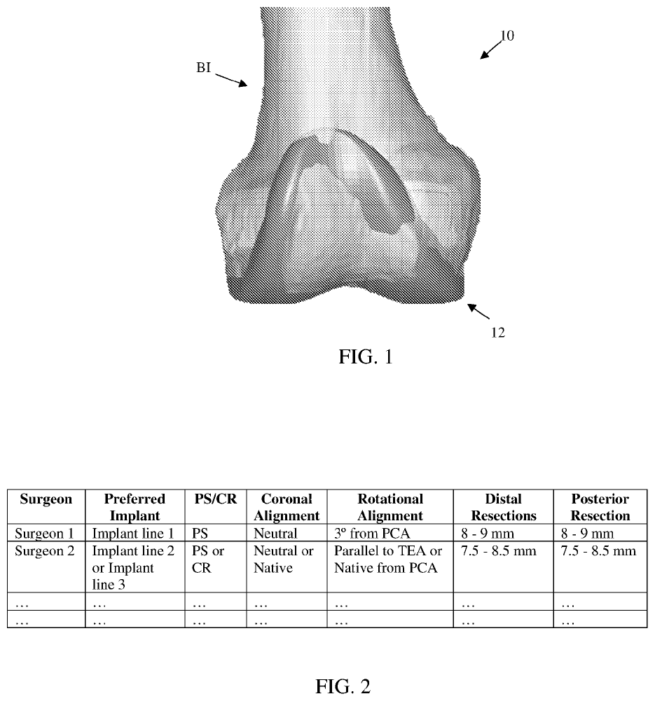

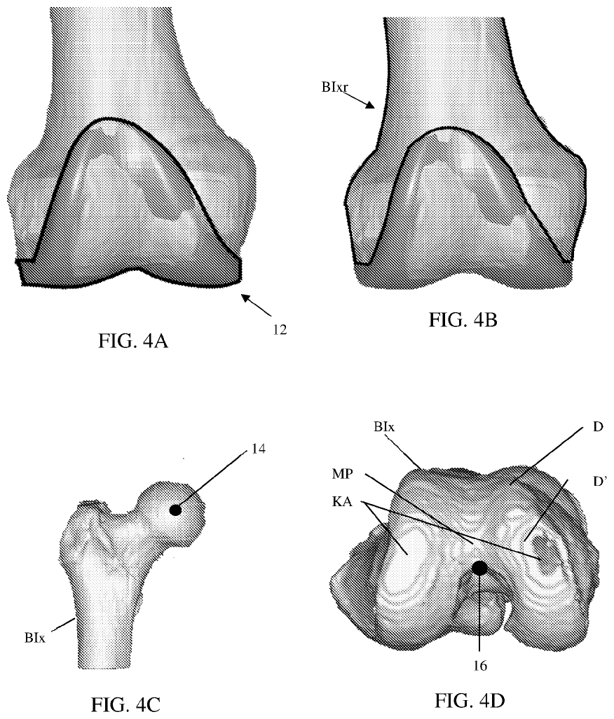

The machine learning program may be based on, for example, TensorFlow 1 or TensorFlow 2, developed by Google Brain, which is an open-source software library for machine learning and artificial intelligence, and adapted to generate the planning models (112a, 112b) described herein. Examples of machine learning algorithms capable of generating the planning models (112a, 112b) illustratively include: random forest (RF), convolutional neural networks (CNNs), random sample consensus (RANSAC), linear support vector machine (LSVM), and ANN. A sub-set of surgical plans from the collection of surgical plans (108a, 108b) may be annotated to train the planning models (112a, 112b) in the machine learning program 110. The input to train the planning models (112a, 112b) may include: an implant image 12 POSED with respect to a remaining bone image “BIxr”; the original bone image “BIx”; the implant image 12; the POSE of the cut surfaces to be formed on the remaining bone “BIxr”; the raw POSE data for the implant POSED with respect to the bone; and/or the planned clinical alignment data for the implant POSED with respect to the bone. With reference to FIGS. 4A to 4D, examples of annotations on one or more images may include: (a) labeling the implant image 12, or portions thereof (e.g., contact surfaces), as shown in FIG. 4A (depicted as the outline of the implant image 12 with respect to the remaining bone image “BIxr”); (b) labelling the remaining bone “BIxr”, or portions thereof (e.g., cut surfaces) resulting from the intersection of the implant image 12 with the original bone image “BIx” as shown in FIG. 4B (depicted as the outline of the remaining bone around the implant image 12); (c) labelling anatomical landmarks that the historical user may have identified on the original bone image “BIx”, or newly identified in the surgical plan (e.g., FIG. 4C. depicts the femoral head center 14 anatomical landmark on a bone image “BIx” of a femur, and FIG. 4D depicts the intercondylar notch 16 anatomical landmark on a bone image “BIx” of a femur); (d) localized bone densities, D and D′; (d) presence of osteophytes; (e) locations and/or a characterization of irregular bone geometry; (f) cartilage thickness; (g) the degree of curvature of one or more portions of bone in the bone image “BIx” (e.g., degree of curvature of the medial or lateral condyle); (h) anatomical references (e.g., mechanical axis of the femur/knee, longitudinal axis of the tibia, epicondylar axis) determined from the bone image “BIx”, the remaining bone shown in the surgical plan, and/or the labelled anatomical landmarks; (i) geometry and/or dimensions of the implant from the implant image 12 in the surgical plan or a stand-alone implant image 12 (e.g., locations of the contact surfaces of the implant, angles between the contact surfaces, medial and lateral distal thickness, medial and lateral posterior thickness, medial and lateral anterior thickness, degree of curvature of an outer surface, location of keel, keel dimensions, etc.); and/or (j) kinematically alignment (KA) points or medial pivot (MP) points (MP). It is appreciated that the KA approach restores the native joint line and limb alignment while medial pivot points achieve a ball and socket joint alignment in the medial compartment of the joint, synonymously known as the medial pivot knee design. Other anatomical landmarks that may be annotated on a bone image “BIx” of a femur may include the medial epicondyle, lateral epicondyle, anterolateral trochlear ridge, anteromedial trochlear ridge, most posterior point on medial condyle, most posterior point on lateral condyle, most distal point on medial condyle, most distal point on lateral condyle, and knee center. Anatomical landmarks that may be annotated on a bone image of a tibia include: midpoint between tibial splines, ankle center, center of medial plateau 128c, center of lateral plateau, tibia tubercle (medial ⅓rd), antero-lateral face, and antero-medial face. The machine learning program 110 may receive additional inputs to train the planning models (112a, 112b), illustratively including: (i) the historical user's planning strategy (e.g., the planning preferences shown in the table in FIG. 1); (ii) a series of implant geometry data (e.g., implant images or raw implant geometry data), each implant geometry data corresponding to an implant of a different implant size for comparison to the implant geometry data the historical user chose for a particular bone image (“BIx”, “BIy”) in a surgical plan; (iii) a library of implant geometry data, the library containing implant geometry data for different implants manufactured by different manufacturers and their associated implant sizes, where the library of implant geometry data are used for comparison to the implant image the historical user chose for a particular bone image (“BIx”, “BIy”) in a surgical plan; and in still other embodiments of the present invention illustratively include (iv) counterexamples for a given historical user; (v) post-surgical feedback from the historical user (e.g., after a procedure, the historical user may provide feedback in the form of adjustments to the surgical plan based on data obtained during and/or after a surgical procedure); and (v) sensor input from a kinematic sensor or an intercompartmental pressure sensor; or combinations thereof.

Some embodiments of the present invention utilize a machine learning processing circuit that processes data obtained and/or reported during pre-operative surgery of patients. According to some embodiments, the machine learning circuits additionally process data obtained from intra-operative and/or post-operative stages of surgery for patients. Over time, the machine learning processing circuit trains a machine learning model based on historical correlations and/or other trends determined between, for example, the variables (metrics or other data) that have been selected by surgeons during the pre-operative stage, the tracked movements during navigated surgery, and the resulting outcomes for patients. The training can include adapting rules of an artificial intelligence (AI) algorithm, rules of one or more sets of decision operations, and/or weights and/or firing thresholds of nodes of a neural network mode, to drive one or more defined key performance surgical outcomes toward one or more defined thresholds or other rule(s) being satisfied. The surgical guidance system processes pre-operative data for a new patent's characteristics through the machine learning model to provide a surgical plan, which may include navigated guidance to a surgeon during the pre-operative stage when generating a surgical plan with implant selection. According to some embodiments, the surgical plan can be provided to a computer-assisted surgical device, such as a surgical robot to execute the surgical plan or navigation system to provide guidance to the surgeon during the intra-operative stage to assist the surgeon with execution of the surgical plan, or the surgical plan may be provided to the surgeon as a workflow task list for the surgeon to execute the surgery according to the surgical plan. Additionally, the surgical plan can be provided to a robot surgery system to control movements of a robot arm that assists the surgeon during execution of the surgical plan.

According to embodiments, the machine learning model that creates a planning model for a particular experienced user is trained over time, as it receives feedback data from more and more planned surgical cases, thereby growing the number of historical cases considered and further refining the planning model. However, it is also understood that the machine learning model and resulting planning model are not adapted from a zero knowledge starting point. Instead, the machine learning model is pre-programmed based on previously planned surgical cases by a particular historical user, as shown in FIG. 3.

According to some embodiments, the planning model is configured to be refined with post-operative data as part of a feedback training component that is configured to obtain post-operative feedback data provided by distributed networked computers regarding surgical outcomes for a plurality of patients, and to train a machine learning model based on the post-operative feedback data.

The machine learning model for generating each planning model includes an AI-powered algorithm component and/or neural network component that is trained to identify correlations between pre-operative stage data, and according to some embodiments intra-operative stage data and/or post-operative stage data.

In some embodiments, a planning model is created by training a machine learning model based on the pre-operative stage data in the form of a collection of previously planned surgical cases. According to embodiments, this data can include any one or more of: patient demographics (e.g., age, gender, BMI, race, comorbidities); patient medical history; and medical image analysis (e.g., the POSE of an implant image with respect to a bone image, implant image data, bone image data, etc.), as described above.

According to embodiments, the machine learning model generates a planning model by analyzing the collection of previously planned surgical cases to recognize patterns in a historical user's decision making process. The machine learning model may recognize patterns regarding any one or more of:
(1) pre-operatively planned or intra-operatively used (“planned or used”) procedure type;
(2) planned or used type of implant(s);
(3) planned or used implant dimension sizing;
(4) planned or used volume of bone graft, or other adjuncts, used with an implant;
(5) planned or used implant location placement and configuration relative to an existing bone (e.g., a planned POSE for an implant with respect to a bone according to a patient's unique bone geometry, surgeon planning strategy, surgeon preferences, and patient factors as described above);
(6) planned or used types of tool(s) and may include planned or used trajectory relative to patient and planned movements;
(7) planned or used incision location on patient;
(8) planned or used cannula insertion path relative to patient;
(9) planned or used retractor configuration;
(10) planned or used retractor operation to obtain access to target location;
(11) deviations between planned and used procedures;
(12) deviations between planned and used implant characteristics (e.g., deviation of an implant device size that is implanted into a patient during surgery from an implant device size defined by a surgical plan);
(13) deviations between planned and used implant positioning and/or insertion trajectory (e.g., data indicating deviation of implant device pose after implantation into a patient during surgery from an implant device pose defined by a pre-operative surgical plan);
(14) deviations between planned and intra-operatively achieved levels of joint correction; and
(15) surgery events (e.g., problems, failures, errors, observations during the surgical procedure).
(16) deviations between: (i) a planned POSE for an implant with respect to a bone having first anatomy characteristics (e.g., bone geometry; bone quality or density and location of said quality or density; location and/or quality of a type of bone (e.g., cortical vs. trabecular); presence of abnormalities (e.g., osteophytes); soft tissue locations & quality (e.g., tendons & ligament locations/quality, cartilage thickness/quality); adjacent bone geometry, quality, or density); and (ii) a planned POSE for an implant with respect to a bone having second anatomy characteristics, where the second anatomy characteristics differs by at least one characteristic from the first anatomy characteristics.
(17) deviations between: (i) a planned POSE for a first implant (e.g., implant size ‘x’) with respect to a bone having first anatomy characteristics; and (ii) a planned POSE for a second implant with respect to a bone having second anatomy characteristics, where the first implant differs from the second implant and the second anatomy characteristics differs by at least one characteristic from the first anatomy characteristics.

In some additional or alternative embodiments, the planning model is are further refined using a feedback training component of the machine learning model based on the post-operative stage data (also called “post-operative feedback data”), which may include any one or more of: patient reported outcome measures; measured outcomes (e.g., deformity correction measurements, Range of Motion (ROM) test, soft tissue balance measurements, kinematics measurements, curvature measurements, other functional outcomes); logged surgery events; and observation metrics. The logged surgery event can include timing, problems (e.g., deviation of robot axes positions from plan, deviation of end effector positions from plan, deviation of surgical tool positions from plan, deviation of implant device position from plan, deviation of implant fit from predicted, unplanned user repositioning of robot arm, deviation of action tool motion from plan, unplanned surgical steps, etc.), failures (e.g., surgeon prematurely stops use of surgical implement before plan completion, etc.), and errors. Some post-operative stage data may be collected using the planning workstation or mobile application (e.g., smartphone or other computer application) that can operate standalone or can be communicatively connected (e.g., WiFi or Bluetooth paired) with one or more patient wearable devices for systematic data collection (functional data and Patient-reported Outcome Measures PROMs) before and after surgery. Sensors operative herein include a kinematic sensor (K A Gustke, et al., J Arthroplasty. 2017; 32(7):2127-2132) or an intercompartmental pressure sensor insert (VERASENSE®) of OrthoSensor. It is appreciated that the present invention in providing a procedure planning is distinct from achieving balance and alignment in a TKA. MA Verstraete et al. Bone Joint Open 2020; 1-6:236-244.

The machine learning model for training a planning model may process the pre-operative stage data, intra-operative stage data, and/or post-operative stage data to form subsets of the data having similarities that satisfy a defined rule. Within each of the subsets, the machine learning model can identify correlations among at least some values of the data, and then train the planning models based on the correlations identified and recognized patterns for each of the subsets. The training can operate to adapt rules of an AI algorithm, rules of one or more sets of decision operations, and/or weights and/or firing thresholds of nodes of a neural network based on the identified correlations to drive one or more outputs (e.g., surgical plan(s)) of the machine learning model toward one or more defined thresholds or other rule(s) being satisfied (e.g., defined key performance surgical outcomes indicated by the post-operative stage data).

More particularly, the machine learning model finds similarities (a threshold level of correlation) among the sets of data obtained for a set of the previous patients and identify what has been learned to be the best surgical plan that has been known to be used for one or more prior surgical patients among that set of previous patients for a particular historical user. Elements in the sets of data may have different weightings based on a defined or learned level of effect in the process to generate a surgical plan that will achieve the best surgical outcome for a patient based on the experience and planning philosophy of the particular historical user for a particular planning model.

The automated planning software 116 is now configured to generate surgical plans like a historical user. The automated planning software 116 may receive a selection of a historical user to generate a surgical plan on a bone image “BIn” of a new patient like the selected historical user. The selection may be made by one of the following: (i) a current user 114; (ii) automatically by the automated planning software 116; or (iii) the automated planning software 116 may be customized to always generate a surgical plan like a particular historical user. Then, based on the selection or customization, the automated planning software 116 executes the corresponding historical user's planning model using at least a first input and a second input. The first input may include a bone image “BIn” of a new patient, such as: (a) an image data set of the bone acquired via computed tomography (CT), magnetic resonance imaging (MRI), ultrasound, x-ray, laser scan, etc.; (b) a 3-D bone model; or (c) a point cloud of the bone. The second input may include at least one of the following: (a) implant geometry data (e.g., an implant image); (b) a series of implant geometry data (e.g., a series of implant images), each implant geometry data corresponding to an implant size (e.g., manufacturer A's implant ranging in size from 1 to 9); or (c) a library of implant geometry data (e.g., a library of implant images), each implant geometry data corresponding to a particular manufacturer's implant and its implant sizes (e.g., manufacturer A's implant ranging in size from 1 to 9, manufacturer B's implant ranging in size from 1 to 6, etc.). The output of the historical user's planning model is a planned POSE for an implant with respect to a bone in the same or nearly the same location as the selected historical user would POSE the implant with respect to the bone. The output may be represented or provided as one or more of the following: (a) an implant image 12 POSED with respect to a remaining bone image “BInr”; (b) the POSE for the cut surfaces to be formed on the remaining bone for mounting an implant thereon in a planned POSE; (c) raw POSE data for an implant POSED with respect to the bone; and/or (d) the planned clinical alignment data for the implant POSED with respect to the bone. Any of the aforementioned (a), (b), (c) and (d), either alone or in combination, may be referred to herein as implant positioning data defined with respect to the bone image. The output may further include software instructions for a computer-assisted surgical device to form the cut surfaces on the remaining bone to a mount an implant thereon in the planned POSE.

The planning models (112a, 112b) may be trained to perform one or more of the following: (i) segment a set of 2-D images of the bone to generate a 3-D bone model as if the selected historical user segmented the same set of 2-D images; (ii) identify anatomical landmarks and anatomical references; (iii) determine a POSE for an implant with respect to a bone; (iv) determine an implant size that the selected historical user would have chosen for the bone image “BIn” (e.g., determine implant size 3 and POSE an implant image of implant size 3 with respect to the bone image “BIn”; (v) determine a particular manufacturer's implant that the selected historical user would have chosen for the bone image “BIn” (e.g., determine manufacturer A's implant as opposed to manufacturer B's implant and POSE an implant image of manufacturer A's implant with respect to the bone image “BIn”); (vi) determine both a manufacturer's implant and implant size that the selected historical user would have chosen for the bone image “BIn” (e.g., determine manufacturer A's implant as opposed to manufacturer B's implant, determine implant size 5 of manufacturer's A's implant, and POSE an implant image of manufacturer A's implant size 5 with respect to the bone image “BIn”); and (vii) POSE an implant with respect to a bone image “BIn” for revision procedures, where the bone image “BIn” includes metal artifacts caused by the imaging (e.g., CT scan) of an implant (e.g., a primary implant) mounted on the bone prior to the imaging.

In some inventive embodiments, the limitations of a computer to perform inductive inference based on a finite set of examples from a given surgeon is addressed by inclusion of surgical counterexamples. In this instance, counterexamples are procedures the surgeon to be modeled would like excluded for personal reasons. As a result, with the inclusion of surgical counterexamples represented as Boolean expressions the ML algorithm learning is enhanced, resulting in a better application of a surgeons preferences to the procedure being planned. In still other embodiments, through resort to a quantum computer performing the inductive inference based on a finite set of examples associated with ML, the computational limitations of binary computing associated with a digital computer are overcome thereby allowing for improved modeling and procedure planning.

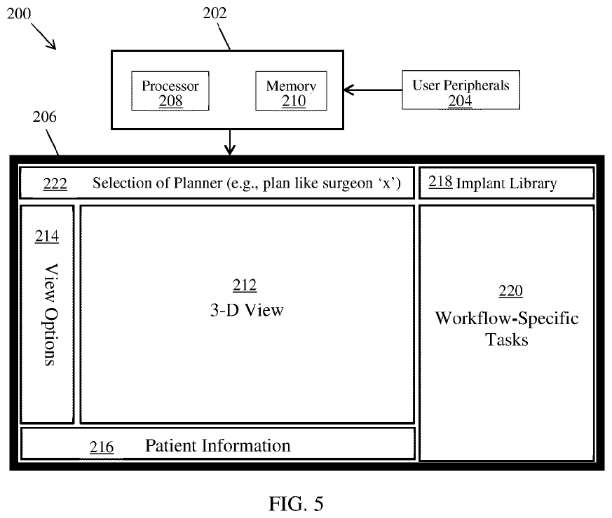

FIG. 5 depicts an embodiment of a TKA planning workstation 200. The workstation 200 includes a computer 202, user-peripherals 204, and a monitor displaying a graphical user interface (GUI) 206. The computer 202 includes a processor 208 operatively coupled to non-transient memory 210 and operating the automatic planning software 116 to execute the planning methods described herein. The user peripherals 204 allow a user to interact with the GUI 206 and may include user input mechanisms such as a keyboard and mouse, or the monitor may have touchscreen capabilities. The GUI 206 may include a three-dimensional (3-D) view window 212 with a rotatable view of a bone, an implant, or other feature of interest. The window 212 including the majority of the area of the GUI 206. In still other embodiments, the window 212 includes the central region of the GUI 206. The GUI 206 also includes at least one of view options window 214, a patient information window 216, an implant library window 218, a workflow-specific tasks window 220, and a historical user selection window 222 surrounding the window 212. Each GUI window can be summarized as follows. The 3-D view window 212 allows the user to view and interact with images (e.g., 3-D bone models, 3-D implant models). The view options window 214 provides widgets to allow the user to quickly change the view of the images, anatomical landmarks, or anatomical references (e.g., mechanical axes, distal condylar plane, posterior condylar plane, tibial plateau plane). The user is able in some inventive embodiments to annotate the images. The patient information window 216 displays the patient's information such as name, identification number, gender, surgical procedure, and operating side (e.g., left femur, right femur). In some inventive embodiments, the user can annotate or otherwise revise the patient information. The implant library window 218 may provide a drop-down menu to allow the user, or planning software, to select a particular implant (e.g., manufacturer A's implant in implant size 2) from a library of implants, and upon selection an implant image of the selected implant is display in the 3-D view window 212. The workflow-specific tasks window 220 includes various widgets to provide several functions illustratively including: guiding the user throughout different stages of the planning procedure; providing an option to select a coronal or rotational alignment goal from a set of alignment goals, such as the coronal and rotational alignment goals shown in the table of FIG. 1; providing tools to adjust the POSE of an implant image with respect to a bone image in desired clinical directions (e.g., medial-lateral direction, flexion-extension rotation, proximal-distal direction); displaying measured values of the alignment and position of the implant image with respect to the bone image (e.g., implant image is 2° from the mechanical axis); and displaying a summary of the surgical plan. The historical user selection window 122 provides an option (e.g., in a drop-down menu) to select a historical user from a set of historical users for automatically generating a surgical plan like the selected historical user. It should be appreciated that not all of the above windows be present in the planning software 116 or viewable on the GUI 206.

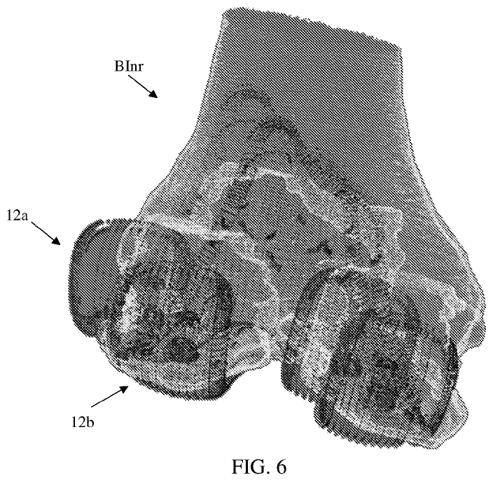

In a particular embodiment, with reference to FIG. 6, the automated planning software 116 may generate an initial surgical plan using conventional techniques, such as the techniques described in U.S. patent application Ser. No. 16/080,735, where an implant image is automatically POSED with respect to a bone image “BIn” using the tools in the implant family window 118 and the workflow-specific tasks window 120 (e.g., by identifying anatomical landmarks, and then selecting a coronal alignment goal and a rotational alignment goal). The initial surgical plan provides an initial POSE for the implant image 12a with respect to the bone image “BIn”. The initial surgical plan is then provided as input into the planning model (112a or 112b) of the selected historical user to automatically generate a second surgical plan. The second surgical plan may have a new POSE for the implant image 12b with respect to the bone image “BIn” that aligns with the planning strategy of the selected historical user.

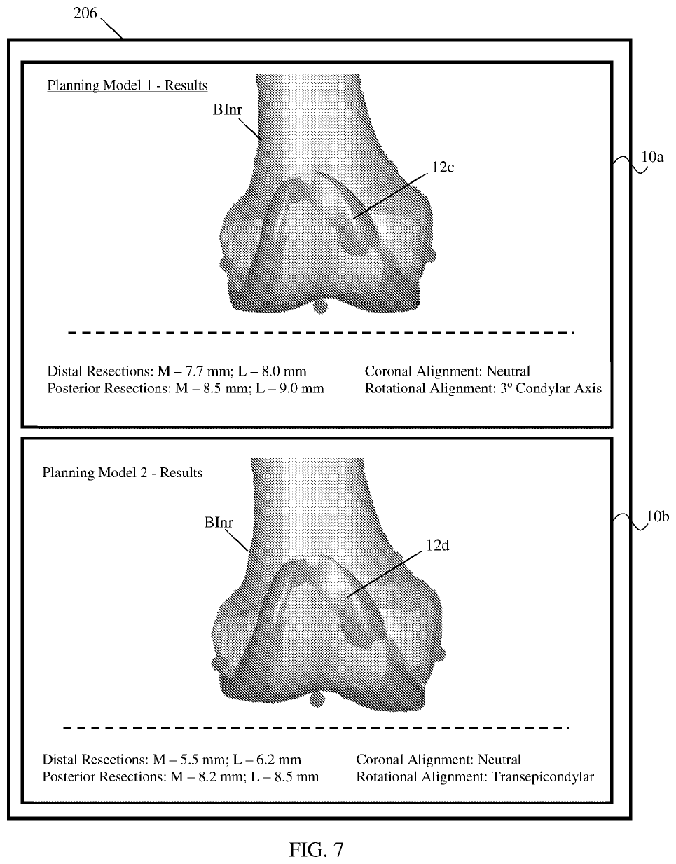

With reference to FIG. 7, the automated planning software 116 may be configured to present two or more surgical plans for comparison. FIG. 7 depicts the results of a first surgical plan 10a and a second surgical plan 10b displayed on the GUI 206. The first surgical plan 10a includes a first implant image 12c in a first POSE with respect to a bone image “BIn”, and the second surgical plan 10b includes a second implant image 12d in a second POSE with respect to the bone image “BIn”. The first surgical plan 10a was generated by planning model 1 and the second surgical plan 10b was generated by planning model 2. This may allow a user to compare how different historical users would generate a surgical plan on a new bone image “BIn”. Here, planning model 1, 112a, (trained to plan like historical user 1) selected a first implant image 12c (e.g., manufacturer A's implant in implant size 4) and POSED the first implant image 12c with respect to the bone image “BIn” according to the following: medial distal resection of 7.7 mm, lateral distal resection of 8.0 mm, medial posterior resection of 8.5 mm, lateral posterior resection of 9.0 mm, neutral mechanical axis coronal alignment, and 3° from the posterior condylar axis in internal-external rotational alignment. Planning model 2, 112b, (trained to plan like historical user 2) selected a second implant image 12d (e.g., manufacturer B's implant in implant size 5) and POSED the second implant image 12d with respect to the bone image “BIn” according to the following: medial distal resection of 5.5 mm, lateral distal resection of 6.2 mm, medial posterior resection of 8.2 mm, lateral posterior resection of 8.5 mm, neutral mechanical axis coronal alignment, and parallel to the transepicondylar axis in internal-external rotational alignment. A current user may review these two surgical plans (10a, 10b) (or more) for the purpose of training, and/or select one (e.g., select the first surgical plan 12c) to form cut surfaces on the patient's remaining bone to mount an implant thereon in the POSE according to the selected surgical plan. It should be appreciated that the bone image “BIn” in the first surgical plan 10a and the second surgical plan 10b is from the same patient but may slightly differ if the planning models (112a, 112b) are also trained to segment images like the respective historical user (102a, 102b) as described below. It should be further appreciated, that the first surgical plan and second surgical plan may be compared absent of any images where a user can compare the one or more of the following: the raw POSE data for an implant POSED with respect to the bone; and/or the planned clinical alignment data for the implant POSED with respect to the bone.

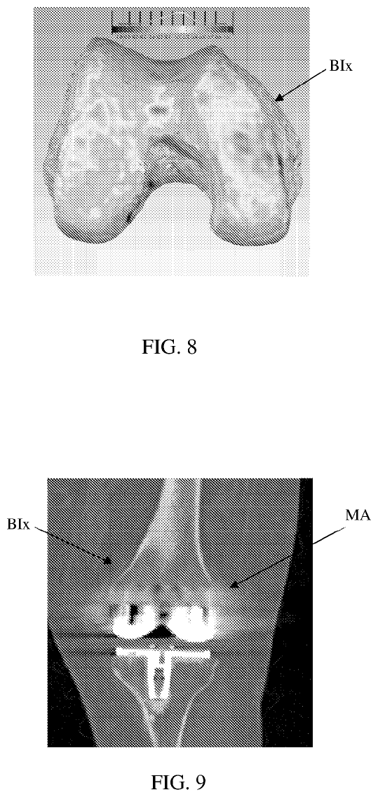

With reference to FIG. 8, the planning models (112a, 112b) may be trained to segment a set of 2-D images of a patient to generate 3-D bone models of the patient, where the planning models are trained using a sub-set of surgical plans from the collection of surgical plans (108a, 108b) from each historical user (102a, 102b). In a particular embodiment, the sub-set of surgical plans are annotated to label certain areas on a bone image (“BIx”, “BIy”) that are more prone to segmentation errors (i.e., “error-prone areas”). An error map may be generated on the bone image (“BIx”, “BIy”) to identify these error-prone areas in each surgical plan from the sub-set of surgical plans, where FIG. 8 depicts an example of an error map for a particular bone image “BIx”. The location of these error-prone areas may be consistent from patient to patient. Therefore, the error-prone areas may be annotated with special attention to fine-tune the segmentation training in the planning models (112a, 112b).

With reference to FIG. 9, the planning models (112a, 112b) may be trained to generate surgical plans for revision cases, where the planning models (112a, 112b) are trained to segment a set of 2-D images “BIx” of a patient that may contain metal artifacts “MA” due to the imaging (e.g., CT scanning) of a bone having an implant already mounted thereon. The planning models (112a, 112b) may be trained on a sub-set of surgical plans from the collection of surgical plans (108a, 108b), where each surgical plan in the sub-set of surgical plans contains metal artifacts in the bone images “BIx”. In other embodiments, the planning models (112a, 112b) may be trained on bone images with simulated metal artifacts to reduce the amount of real bone images containing metal artifacts. The planning models (112a, 112b) may therefore generate a 3-D bone model of a patient's bone from a set of 2-D images containing metal artifacts, and subsequently generate a new surgical plan for that patient in revision cases.

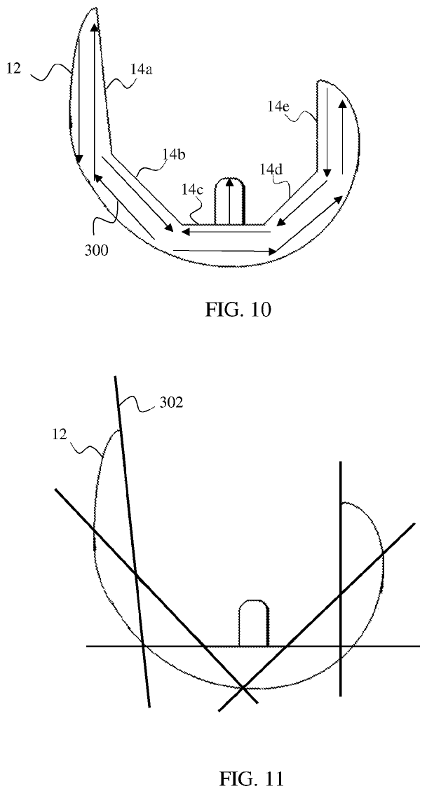

With reference to FIGS. 10 and 11, software instructions may be included or associated with implant geometry data (e.g., an implant image 12), where the software instructions direct a computer-assisted surgical device to assist in the formation of cut surfaces (either directly or indirectly) on the remaining bone to mount an implant thereon in the same POSE defined in the surgical plan. Examples of “software instructions” include a cut-file, virtual boundaries, virtual paths, or virtual planes. A “cut-file” may include instructions (e.g., end-effector cut paths 300 (as shown in FIG. 10), points, orientations, feed rates, or spindle speeds, and any combination thereof as well as other factors) that direct the CAS device during the formation of the cut surfaces on the bone automatically (e.g., a surgical robot executes the instructions to control movement of an end-effector). The “cut-file” instructions or portions thereof may be generated based on the implant geometry data (e.g., the geometry of an implant image 12, raw implant dimensions) and may be generated with the aid of computer-aided manufacturing (CAM) software. FIG. 10 depicts a plurality of cut paths 300 defined at locations with respect to at least a portion of the geometry of an implant image 12 (e.g., a femoral implant image for TKA). A CAS device is directed to automatically follow these cut paths 300 to directly form the cut surfaces on the remaining bone, and therefore forms the cut surfaces according to the POSE of the implant image positioned with respect to the bone image in the surgical plan. More specifically, the cut surfaces are formed at locations on the remaining bone corresponding to the implant surfaces (14a, 14b, 14c, 14d, 14e) of the implant image 12 as POSED with respect to the bone image in the surgical plan. In another embodiment, with reference to FIG. 11, the “software instructions” may be virtual boundaries 302 defined at locations with respect to the implant image 12 (an more specifically the implant surfaces) which direct a CAS device to provide feedback (e.g., active, semi-active, haptic, or power control) to a user to assist in the prevention of cutting bone beyond the virtual boundaries 302 while the user manually maneuvers an end-effector of the CAS device during the formation of the cut surfaces. In a further embodiment, the “software instructions' may be virtual paths defined at locations with respect to the implant image 12, which direct a CAS device to provide feedback (active, semi-active, haptic, or power control) to a user to assist in maintaining an end-effector of the CAS device along the virtual path while the user maneuvers the end-effector during the formation of the cut surfaces.

In a further embodiment, the “software instructions” may be one or more virtual planes defined at locations with respect to at least a portion of the geometry of a cut guide or alignment guide, which directs a CAS device to align pins coincident with the virtual plane for insertion of the pins in the bone. The cut guide or alignment is then placed on the pins for guiding a cutting tool in the formation of the one or more cut surfaces as further described in U.S. patent application Ser. No. 15/778,811, assigned to the assignee of the present application, and incorporated by reference herein in its entirety.

Examples for Generating a Surgical Plan with a Trained Planning Model

Example 1: A current user 114 is assigned a new patient case and uploads a set of 2-D images of the patient acquired via a CT scan to the automated planning software 116. The current user 114 segments the 2-D images in the automated planning software 116 using image segmenting tools and techniques known in the art to generate a 3-D bone model of the patient. The current user 114 then selects historical user 1 to generate a surgical plan like historical user 1. The automated planning software 116 then executes planning model 1 using a first input and a second input, where the first input is the 3-D bone model, and the second input is a set of 3-D implant models corresponding to manufacturer A's implant ranging in size from 1 to 9. The output of planning model 1 is surgical plan 10b as shown in FIG. 7 and described above, having a 3-D implant model in size 4 and POSED with respect to a 3-D bone model. The current user 114 saves the surgical plan 10a and transfers the surgical plan 10a to an autonomous surgical robot in the operating room (OR). The autonomous surgical robot executes a cut-file (having cut-paths defined at locations with respect to at least a portion of the geometry of the 3-D bone model) to automatically form the cut surfaces on the remaining bone to mount an implant thereon in the same POSE as designated in the surgical plan.

Example 2: A current user 114 is assigned a new patient case and uploads a set of 2-D images of the patient's acquired via a CT scan to the automated planning software 116. The current user 114 then selects historical user 2 to generate a surgical plan like historical user 2. The automated planning software 116 then executes planning model 2 using a first input and a second input, where the first input is the set of 2-D images of the patient, and the second input is a library of 3-D implant models corresponding to manufacturer A's implant ranging in size from 1 to 9 and manufacturer B's implant ranging in size from 0 to 6. Planning model 2 segments the bone from the set of 2-D images of the patient, and outputs surgical plan 10b as shown in FIG. 7 and described above, having a 3-D implant model from manufacturer B in size 6 and POSED with respect to a 3-D bone model. The current user 114 saves the surgical plan 10b and transfers the surgical plan 10b to a haptic surgical robot in the operating room (OR). The haptic surgical robot executes software instructions (having virtual boundaries defined at locations with respect to at least a portion of the geometry of the 3-D implant model) to provide feedback (e.g., active, semi-active, haptic, or power control) to a user to assist in the prevention of cutting bone beyond the virtual boundaries 302 while the user manually maneuvers an end-effector of the haptic surgical robot device during the formation of the cut surfaces.

1. A surgical planning system for planning a surgery based on experience of a historical user, the system comprising:
a computer operatively coupled to a display for displaying a graphical user interface (GUI) and a processor configured to execute the planning software operative to:
receive a bone image and display the bone image on the GUI;
receive, via the GUI, a selection of a planning model corresponding to experience of a first historical user, the planning model based on recognized patterns in a collection of planned cases of the first historical user;
analyze the bone image of the patient to determine a set of characteristics of the bone image;
compare the set of characteristics of the bone image to bone characteristics of the collection of planned cases; and
generate a surgical plan comprising implant positioning data defined with respect to the bone image based on the set of characteristics of the bone image and the recognized patterns of the planning model corresponding to experience of the first historical user.

2. The surgical planning system of claim 1 wherein the implant positioning data comprise an implant image, wherein the planning software is further operative to select the implant image from a plurality of implant images based on the set of characteristics of the bone image and the recognized patterns of the planning model.

3. The surgical planning system of claim 1 wherein the planning software includes a plurality of planning models, each planning model corresponding to a different historical user.

4. The surgical planning system of claim 3 wherein the planning software is further operative to receive a selection of one or more planning models from the plurality of planning models or provide cutting instructions to direct a computer-assisted surgical device during formation of one or more cut surfaces on the bone for mounting an implant corresponding to the implant image on the one or more cut surfaces in a POSE corresponding to the POSE of the implant image POSED with respect to the bone image.

5. (canceled)

6. (canceled)

7. The surgical planning system of claim 1 further comprising a training computer operative to generate the planning model based on the collection of planned cases from the first historical user.

8. The surgical planning system of any of claim 7 wherein in generating the planning model, the training computer is operative to receive the collection of planned cases of the first historical user and utilize a machine learning model to recognize patterns in the collection of planned cases of the first historical user alone or in combination with generating an additional plurality of planning models each corresponding to experience of different historical users.

9. (canceled)

10. The surgical planning system of claim 1 wherein the planning software is further operative to:
receive, via the GUI, a selection of a second planning model corresponding to a second historical user, the planning model based on recognized patterns in a collection of planned cases of the second historical user;
compare the set of characteristics of the bone image to bone characteristics of the collection of planned cases of the second historical user; and
generate a second surgical plan comprising second implant positioning data defined with respect to the bone image based on the set of characteristics of the bone image and the recognized patterns of the second planning model corresponding to experience of the second historical user.

11. The surgical planning system of claim 10 wherein the second implant positioning data comprises the implant image or a second implant image, where the planning software is further operative to select the implant image or the second implant image from the plurality of implant images based on the set of characteristics of the bone image and the recognized patterns of the second planning model.

12. The surgical planning system of claim 10 wherein the automated planning software is further operative to display the surgical plan and the second surgical plan for comparison.

13. The surgical planning system of claim 1 wherein the GUI comprises a three-dimensional (3-D) view window with a rotatable view of the bone image.

14. The surgical planning system of claim 13 wherein the three-dimensional (3-D) view window covers the majority of an area of the GUI or the central region of the GUI.

15. (canceled)

16. The surgical planning system of claim 13 wherein GUI further comprising at least one of additional window of view options window, a patient information window, an implant library window, a workflow-specific tasks window, a historical user selection window or a combination of any of the aforementioned.

17. (canceled)

18. The surgical planning system of claim 13 wherein the GUI further comprises widgets to allow the user to change at least one of a view of the images, anatomical landmarks, or anatomical references alone or in combination with the GUI is configured to allow the user to annotate the images, annotate the patient information or otherwise revise the patient information, or a combination thereof.

19. (canceled)

20. The surgical planning system of claim 16 wherein the implant library window is present and further comprises a drop-down menu to allow the user or planning software to select a particular implant from a library of implants alone, or in combination with the particular implant from the library of implants is displayed in the 3-D view window upon selection.

21. (canceled)

22. The surgical planning system of claim 16 wherein the implant library window is present and further comprises widgets to provide several functions including at least guiding the user throughout different stages of the planning procedure, providing an option to select a coronal or rotational alignment goal from a set of alignment goals, providing tools to adjust the POSE of an implant image with respect to a bone image in desired clinical directions, displaying measured values of the alignment and position of the implant image with respect to the bone image, and displaying a summary of the surgical plan.

23. A surgical planning system for planning a surgery based on experience of a historical user, the system comprising:
a computer comprising a processor configured to execute planning software, the planning software operative to:
receive a bone image;
receive a selection of a planning model corresponding to experience of a first historical user, the planning model based on recognized patterns in a collection of planned cases of the first historical user;
analyze the bone image to determine a set of characteristics of the bone image;
compare the set of characteristics of the bone image to bone characteristics of the collection of planned cases; and
generate a surgical plan comprising position and orientation (POSE) data for a planned POSE of an implant with respect to a bone based on the set of characteristics of the bone image and the recognized patterns in the planning model.

24. The surgical planning system of claim 23 wherein the implant positioning data defined with respect to the bone image comprises at least one of: (i) an implant image positioned and oriented (POSED) with respect to the bone image in the planned POSE; (ii) a POSE of one or more cut surfaces to be formed on remaining bone for mounting an implant thereon in the planned POSE; and/or (iii) clinical alignment data corresponding to the planned POSE.

25. The surgical planning system of claim 23 wherein the planning software includes a plurality of planning models, each planning model corresponding to a different historical user.

26-30. (canceled)

31. A computerized method for positioning an implant image relative to a bone image, comprising:
providing a first planning model and a second planning model, wherein the first planning model is generated with machine learning using first historical planning data from a first historical user and the second planning model is generated with machine learning using second historical planning data from a second historical user;
receiving a selection of the first planning model or the second planning model; and
executing the selected first planning model or second planning model with an input comprising a bone image to automatically define implant positioning data with respect to the bone image.

32. (canceled)

33. (canceled)
